(19) United States
(12) Patent Application Publication
(10) Pub. No.: US 2005/0075557 A1
(43) Pub. Date: Apr. 7, 2005

(54) Method for drawing three-dimensional image by modeling second object connected to first object

(75) Inventor: Mitsuru Kamiyama, Tokyo (JP)

Correspondence Address: GREENBLUM & BERNSTEIN, P.L.C., 1950 ROLAND CLARKE PLACE, RESTON, VA, 20191, US

(73) Assignee: Kabushiki Kaisha Square Enix, Tokyo (JP)

(21) Appl. No.: 10/912,105

(22) Filed: Aug. 6, 2004

(30) Foreign Application Priority Data
Aug. 6, 2003 (JP) P2003-287885

(51) Int. Cl. A61B 5/05

(52) U.S. Cl. 600/407

(57) ABSTRACT

Hair is connected to a head portion of a character that is formed by multiple polygons. The positions of the respective bones provided to portions other than the hair are calculated based on an operation of the character. A reference axis is fixed based on the positions of the bones of the portions other than the hair. A position of a bone arranged in the hair is calculated based on an external force. When an angle, which the bone of the hair forms with the reference axis, is not in the range of a predetermined reference angle, the position of the bone of the hair is corrected to be in the range of the reference angle. The positions of the polygons, which form the character, are determined based on only the positions of the bones provided to the character.

### CROSS REFERENCE TO RELATED APPLICATION

The present disclosure relates to subject matter contained in Japanese Patent Application No. 2003-287885, filed on Aug. 6, 2003, the disclosure of which is expressly incorporated herein by reference in its entirety.

### BACKGROUND OF THE INVENTION

1. Field of the Invention

The present invention relates to computer graphics, for example in video games. More particularly, the present invention relates to a technique for drawing a three-dimensional image by modeling a first object and a second object that independently operate at portions other than their connection point.

2. Description of the Related Art

A three-dimensional game, which causes an object such as a character and the like to operate in a virtual three-dimensional space and displays an image obtained by perspective-transforming the object by a virtual camera, is common in recent video games. In consideration of the actual world, the object existing in the virtual three-dimensional space must respond to various external forces (for example, wind force). It is an important task in the three-dimensional game to reproduce such an external influence in the operation of the object to create a realistic image.

Unexamined Japanese Patent Publication No. 2003-51030 discloses a method for generating a three-dimensional image that can realistically reproduce a state that clothes, which a character wears, are waving in the wind. In this method, a connection degree where a vertex of each polygon is connected to each bone and a reference amount of movement, which is a maximum value of an amount of movement of a vertex connected for each bone are preset. In this method, an amount of movement of each vertex of each polygon and a direction of movement thereof are obtained based on the set connection degree and reference amount of movement to calculate a position of each polygon.

When the vertex position of the polygon is calculated under the influence of only an external force such as wind by this method, a position, which cannot be present in reality, is calculated as the vertex position of the polygon in some cases. The position, which cannot be present in reality, is a position where a polygon that forms the clothes falls in the body of the character. To avoid this problem, in the field of the computer graphics, there is conventionally known a technique for preventing the polygon from falling in the body of the character by determining collisions using an object for collision detection.

However, this type of collision detection must be performed for each vertex of the polygon and requires extremely large amounts of calculations. In video games, since processing must be executed in real time, when the number of polygons is large, there is a possibility that shortage of calculation time for drawing will occur. In order to avoid this problem, the number of polygons that forms the object may be reduced. However, when the number of polygons is reduced, a curved surface of a drawn image is angulated, resulting in a less realistic image.

In the field of the computer graphics, the operation of the character itself is generally calculated using kinematics and dynamics. In the field of computer graphics, the inverse kinematics and the inverse dynamics are also applied to prevent the character from breaking through a wall and the joint of the character from being bent more than a set amount. However, in the inverse kinematics and the inverse dynamics, the calculation becomes extremely complicated and the number of calculations increases as compared with the kinematics and the dynamics. Though active operations such as walking and running can be calculated by the inverse kinematics and the inverse dynamics, there is a problem that a passive operation such as waving of clothes by the influence of the external force such as wind cannot be appropriately calculated by the inverse kinematics and the inverse dynamics.

### SUMMARY OF THE INVENTION

An object of the present invention is to draw a three-dimensional image with a small amount of calculation as modeling operations of a first object and a second object, which independently operates at portions other than their connection point.

In order to attain the above object, a three-dimensional image drawing apparatus according to a first aspect of the present invention draws a three-dimensional image by modeling a first object and a second object that is connected to the first object and includes a bone defining display positions of multiple polygons. The second object is capable of operating, at a portion other than a connection point, independently of the first object.

The three-dimensional image drawing apparatus includes a bone position calculator that calculates a position of a bone included in the second object when the second object moves according to a predetermined operation condition. The three-dimensional image drawing apparatus further includes a collision detector that determines whether the second object is where no collision with the first object occurs, based upon the calculated position of the bone. The three-dimensional image drawing apparatus further includes a bone position corrector that recalculates the position of the bone included in the second object to be where no collision with the first object occurs when the second object is determined as being where collision with the first object occurs. The three-dimensional image drawing apparatus further includes a polygon position calculator that calculates the position of each of the polygons that form the second object with reference to the position of the bone, which is located where no collision with the first object occurs. The three-dimensional image drawing apparatus further includes an image rendering system that draws the polygons that form the second object according to the position calculated by the polygon position calculator to create an image where the first and second objects are modeled.

In the three-dimensional image drawing apparatus, when the second object is where collision with the first object occurs according to the position of the bone calculated by the bone position calculator, the position of the bone is corrected to where no collision with the first object occurs by the bone position corrector. The position of each polygon that forms the second object is calculated with reference to the position of the bone, which is located where no collision occurs. The position where no collision occurs is calculated to draw each polygon, so that a natural and realistic image is drawn. This eliminates the need for determining collision for each polygon to make it possible to draw the image with a small number of calculations.

In order to attain the above object, a three-dimensional image drawing apparatus according to a second aspect of the present invention draws a three-dimensional image by modeling a first object, which is formed by multiple polygons, and a second object, which is formed by multiple polygons and includes a bone defining the display positions of the multiple polygons. The second object is capable of moving, at a portion, other than a connection point independently of the first object. The three-dimensional image drawing apparatus has a program memory that stores a program and a processor that executes the program.

The program causes the processor to calculate the position of each bone included in the second object when the second object moves according to a predetermined operation condition. The program further causes the processor to determine whether the second object is where no collision with the first object occurs at the calculated position of the bone. The program further causes the processor to recalculate the position of the bone included in the second object to be where no collision with the first object occurs when the second object is determined as being where collision with the first object occurs. The program further causes the processor to calculate the position of each of the polygons that form the second object with reference to the position of the bone, which is located where no collision with the first object occurs. The program further causes the processor to draw the polygons that form the second object according to the calculated position to create an image where the first and second objects are modeled.

The program stored in the memory in the three-dimensional image drawing apparatus according to the second aspect of the present invention can be recorded on a computer-readable storage medium. The computer-readable storage medium may be a storage medium constructed to be removably loaded in the computer apparatus and provided separately from the computer apparatus. The computer-readable storage medium may be a storage medium such as a fixed disc device that is included in the computer apparatus and provided together with the computer apparatus. In the game program stored in the memory of the three-dimensional image drawing apparatus according to the second aspect of the present invention, the data signal can be superimposed on a carrier wave from a server apparatus existing on a network and the result is distributed via the network.

In the three-dimensional image drawing apparatuses according to the first and second aspects, it is possible to use a general-purpose computer such a personal computer, etc., in addition to a dedicated video game apparatus. In the three-dimensional image drawing apparatuses according to the first and second aspects, it is possible to use electronic equipment capable of operating as a computer apparatus such as a cellular phone, etc. Moreover, the apparatuses may be portable or stationary.

In order to attain the above object, a three-dimensional image drawing method according to a third aspect of the present invention draws a three-dimensional image by modeling a first object and a second object that is connected to the first object and includes a bone defining display positions of multiple polygons. The second object is capable of moving, at a portion other than a connection point, independently of the first object.

The three-dimensional image drawing method calculates a position of a bone included in the second object when the second object moves according to a predetermined operation condition. The three-dimensional image drawing method further determines whether the second object is where no collision with the first object occurs at the calculated position of the bone. The three-dimensional image drawing method further recalculates the position of the bone included in the second object to be where no collision with the first object occurs when the second object is determined as being where collision with the first object occurs. The three-dimensional image drawing method further calculates the position of each of the multiple polygons that form the second object with reference to the position of the bone, which is located where no collision with the first object occurs. The three-dimensional image drawing method further draws the polygons that form the second object according to the calculated position to create an image where the first and second objects are modeled.

### BRIEF DESCRIPTION OF THE DRAWINGS

FIG. 1 is a block diagram illustrating a configuration of a video game apparatus to which an embodiment of the present invention is applied;

FIG. 2 is a view illustrating a configuration of a player character used in a three-dimensional video game according to an embodiment of the present invention;

FIGS. 3A and 3B are views explaining movement of hair of the player character of FIG. 2;

FIGS. 4A and 4B are views explaining a reference angle fixed to bones for the hair of the player character of FIG. 2;

FIGS. 5A to 5F are views giving explanations of positioning of bones for the hair of the player character of FIG. 2 and positioning of each polygon;

FIG. 6 is a flowchart illustrating a main processing in a three-dimensional video game according to an embodiment of the present invention;

FIG. 7 is a flowchart specifically illustrating a hair bone position decision processing of FIG. 6; and

FIG. 8 is a view illustrating a configuration of another character used in a three-dimensional video game according to an embodiment of the present invention.

### DETAILED DESCRIPTION OF PREFERRED EMBODIMENT

An embodiment of the present invention will be specifically described with reference to the drawings. This embodiment shows an example of a case in which the present invention is applied to draw an image of a player character in a three-dimensional video game.

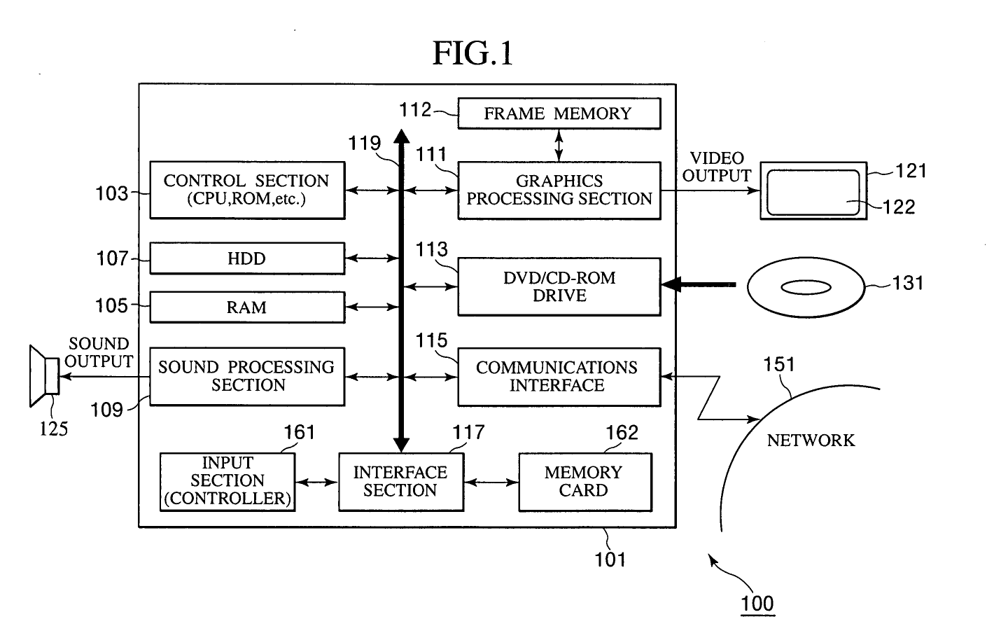

FIG. 1 is a view illustrating a configuration of a video game apparatus 100 that executes a three-dimensional video game according to this embodiment. As illustrated in the figure, the video game apparatus 100 is mainly constructed to include a video game main body 101. The video game main body 101 includes a control section 103, a RAM (Random Access Memory) 105, a hard disk drive (HDD) 107, a sound processor 109, a graphics processor 111, a DVD/CD-ROM drive 113, a communications interface 115, and an interface section 117, which are connected to an internal bus 119.

The sound processor 109 of the video game main body 101 is connected to a sound output device 125, which is a speaker, and the graphics processor 111 is connected to a display device 121 having a display screen 122. A storage medium (DVD-ROM or CD-ROM) 131 can be attached to the DVD/CD-ROM drive 113. The communications interface 115 is connected to a network 151. An input section (controller) 161 and a memory card 162 are connected to the interface section 117.

The control section 103 includes a CPU (Central Processing Unit), a ROM (Read Only Memory), etc., and executes a program stored on the HDD 107 or the storage medium 131 to control the video game apparatus 100. The control section 103 has an internal timer. The RAM 105 is a work area for the control section 103. The HDD 107 is a storage area for storing a program and data. In the case where a program executed by the control section 103 instructs the sound processor 109 to output a sound, the sound processor 109 interprets the instruction and outputs a sound signal to the sound output device 125.

The graphics processor 111 develops an image onto frame memory 112 and outputs a video signal, which displays the image on the display screen 122 of the display device 121 according to a drawing command output from the control section 103. It is assumed that one frame time of the image included in the outputting video signal is, for example, 1/30 sec. The frame memory 112 has areas where images for two frames are developed, and the images are developed alternately in the respective areas for each frame. The DVD/CD-ROM drive 113 reads the program and data from the storage medium 131. The communications interface 11 is connected to the network 151 to perform communications with other computers.

The interface section 117 outputs input data sent from the input section 161 to the RAM 105, and the control section 103 interprets it to carry out arithmetic processing.

The input section 161 includes a directional key and multiple operation keys. The directional key is used to move a character (to be described later) and a cursor. The operation button is used to input a predetermined instruction. The interface section 117 forwards data, indicative of the progress of the game stored in the RAM 105, to the memory card 162 based on the instruction from the control section 103. The interface section 117 reads data of the game from the memory card 162 and transfers the read data to the RAM 105 based on the instruction from the control section.

The program and data for performing the game by the video game apparatus 100 are first stored on, for example, the storage medium 131. The program and data are read by the DVD/CD-ROM drive 113 and loaded onto the RAM 105 at the time of execution. The control section 103 processes the program and data loaded onto the RAM 105, outputs a drawing command to the graphics processor 111, and outputs an instruction of a sound output to the sound processor 109. Intermediate data is stored in the RAM 105 while the control section 103 performs processing.

In the three-dimensional video game according to this embodiment, a player operates the input section 161 to move a player character on a field formed in a virtual three-dimensional space, thereby the game is progressed. The virtual three-dimensional space where the field is formed is positioned by a world coordinate system. The field is structured by multiple surfaces, and coordinates of vertexes of the respective structural surfaces are fixed as characteristic points.

A state when wind blows is simulated in the three-dimensional space in which the player character moves. For example, a wind direction and a wind speed for each frame are decided according to a random number obtained for each frame and data of wind direction and wind speed relating to previous several frames.

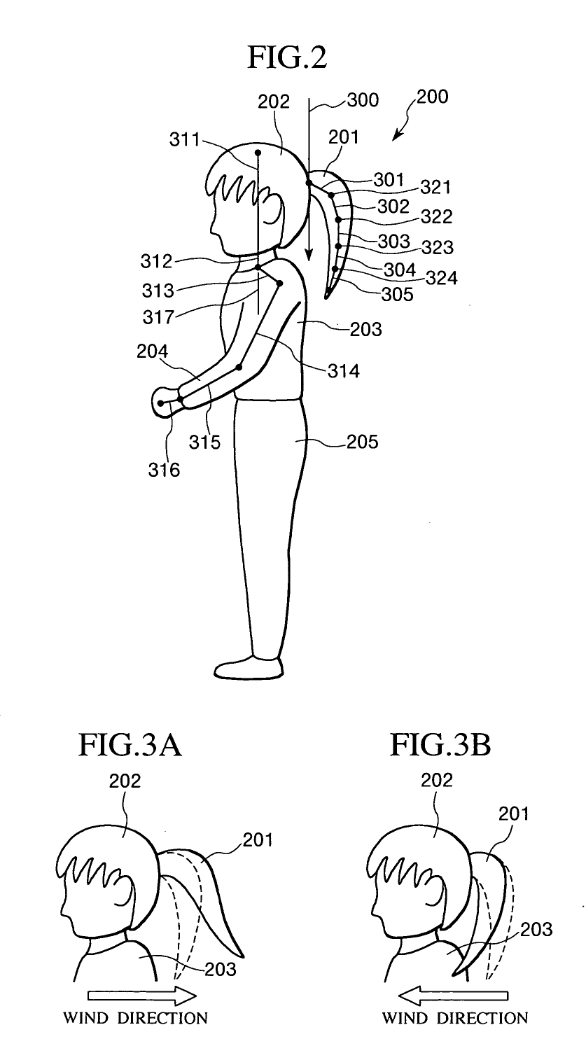

FIG. 2 is a view illustrating a configuration of the player character used in this three-dimensional video game. As illustrated in the figure, a player character 200 includes a head 202, a body 203, an arm 204, and a leg 205. Hair 201 in a ponytail style is added to the head 202. Each portion of the player character 200 includes multiple polygons, and the characteristic point (vertex of each polygon) is fixed by the coordinates in a local coordinate system. The portions of the the hair 201 excluding an end portion connected to the head 202 can be moved independently of the movement of the head 202.

In the player character 200, virtual bones 301 to 305 and 311 to 317 are provided. The position of each polygon that forms the player character 200 is decided by the positions of these bones 301 to 305 and 311 to 317. The positions of bones 311 to 317 provided to the portions other than the hair 201 are decided by only a passive operation of the player character 200. The positions of bones 311 to 317 are decided by a conventional method, such as kinematics and dynamics.

The bones 301 to 305 provided to the hair 201 are connected to one another through joints 321 to 324 sequentially, and operate passively upon receiving an external force instead of the active operation of the player character 200. The external force to be added to the hair 201 includes at least gravity reflected by the weight of the hair 201, a kinetic inertial force generated by the operation of the player character 200, and a force caused by wind flowing in the virtual three-dimensional space. Moreover, there is a case that the amount of force differs for each portion. The weight of the hair 201 is preset based on each of the bones 301 to 305 as a unit in order to calculate the positions of the bones 301 to 305. It is assumed that the weight of the hair 201 is low enough to neglect the influence exerted on the passive operation of the player character 200.

An explanation will be next given of movement of the hair 201 of the player character 200. FIGS. 3A and 3B are views each explaining movement of the hair 201. For example, as illustrated in FIG. 3A, when the player character 200 receives the wind from the front, the hair 201 waves in a backward direction of the head 202 and body 203. As illustrated in FIG. 3B, when the player character 200 receives the wind from the back, the hair 201 waves in the direction of the head 202 and body 203. When the player character 200 runs or stops, the hair 201 is operated passively according to the kinetic inertia.

In order to determine the positions of the bones 301 to 305 provided to the hair 201 as described later, a virtual reference axis 300 is set to the player character 200 every time bone positioning is performed. The reference axis 300 is set on a straight line connecting, for example, the position of a connected portion between the hair 201 and the head 202 to a predetermined position, which is a back side of the body 203. When the player character 200 is moved, the reference axis 300 is also moved.

The positions of the bones 301 to 305 provided to the hair 201 that are moved passively as described above can be also calculated using the conventionally used method based on an external force which is passively received according to the kinetic inertia and wind. In addition, when the positions of the bones 301 to 305 are calculated based on the external force, the polygon, which forms the hair 201 positioned according to the bones 301 to 305, may fall in the interior of the head 202 and the interior of the body 203 (this case is hereinafter called a collision) depending on the magnitude of the external force acting on the hair 201.

In order to prevent occurrence of such a collision, an angle formed by the reference axis 300 and the bone 301, an angle formed by the bone 301 and the bone 302, an angle formed by the bone 302 and the bone 303, an angle formed by the bone 303 and the 304, and an angle formed by the bone 304 and the bone 305 are respectively fixed within predetermined reference angles. In order to determine the reference angle, there is used a coordinate system in which the direction of the reference axis 300 is an x-axial direction, front and back directions of the player character 200 are a y-axial direction, and right and left directions are a z-axial direction. For example, in the case where the positions of the bones 301 to 305 are obtained by a matrix operation using a vector processor, the matrix operation that moves an X axis of the world coordinate system to the reference axis is performed to determine the reference angle and the X axis can be returned to the original world coordinate system by an inverse matrix operation.

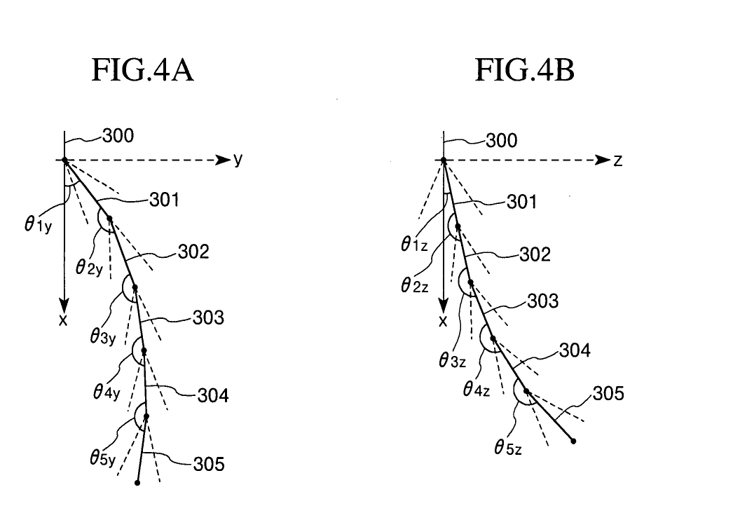

FIGS. 4A and 4B are views each explaining the fixed reference angles of the bones 301 to 305. Since the coordinate system where the direction of the reference axis 300 is used as an x-axial direction, there is no need to determine an x component here. As illustrated in FIG. 4A, reference angles, which are shown by dotted lines, are fixed to a Y component θ1y of an angle formed by the reference axis 300 and the bone 301, a Y component θ2y of an angle formed by the bone 301 and the bone 302, a Y component θ3y of an angle formed by the bone 302 and the bone 303, a Y component θ4y of an angle formed by the bone 303 and the bone 304, and a Y component θ5y of an angle formed by the bone 304 and the bone 305, respectively.

At the time of calculating the positions of the bones 301 to 305, when Y components θ1y, θ2y, θ3y, θ4y, and θ5y of the respective angles exceed the range of the reference angle, the positions of the bones 301, 302, 303, 304 and 305 are corrected to the positions at which reference angles are set. Similarly, as illustrated in FIG. 4B, reference angles, which are shown by dotted lines, are fixed to Z components θ1z, θ2z, θ3z, θ4z, and θ5z of the respective angles. Then, when these Z components exceed the range of the reference angles, the positions of the bones 301, 302, 303, 304 and 305 are corrected to the positions at which reference angles are set.

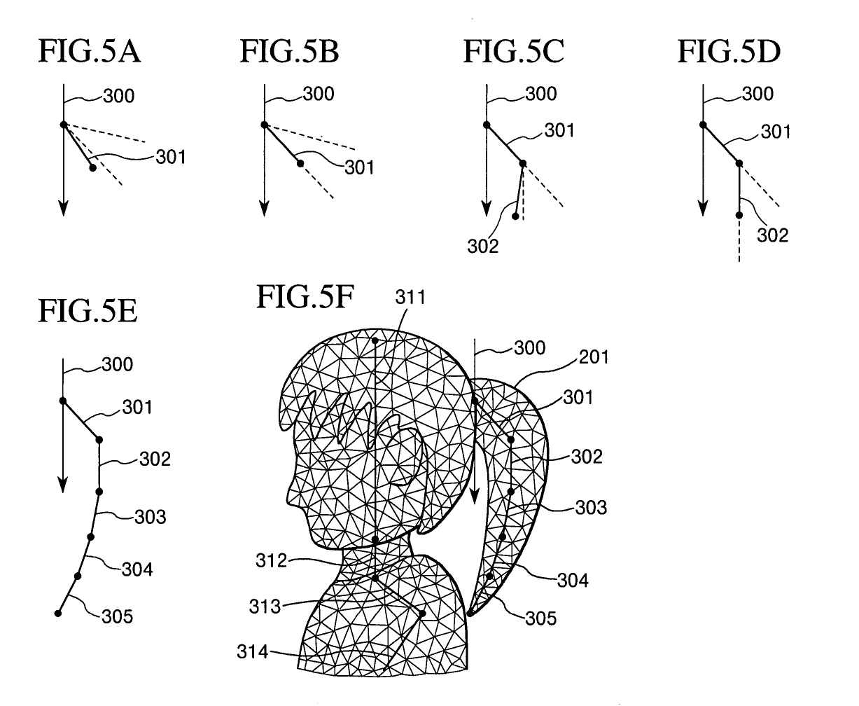

An explanation will be next given of positioning of the bones 301 to 305 and 311 to 317 provided to the player character 200 and positioning of each polygon which forms the hair 201, the head 202, the body 203, the arm 204, and the leg 305. FIGS. 5A to 5F are views each explaining the method for positioning the bones 301 to 305 and 311 to 317 and the positioning of each polygon. Though no explanation is given for separate y components and z components of the respective angles, these components are typically separately calculated.

The positions of the bones 311 to 317 provided to the portions other than the hair 201 are decided by the conventionally used method. When the positions of the bones 311 to 317 are decided, the positions of the polygons, which form the head 202, the body 203, the arm 204, and the leg 305, respectively, are naturally decided. The connected portion between the hair 201 and the head 202 and a predetermined position of the back of the body 203 are obtained, so that the reference axis 300 is set.

When the reference axis 300 is set, the position of the bone 301, which is the closest to the head 202, is calculated according to the external force that the hair 201 receives regardless of the presence of the head 202 and body 203 of the player character 200. Then, an angle θ1 formed by the bone 301 and the reference axis 300 at the calculated position is obtained. As illustrated in FIG. 5A, when the calculated angle θ1 exceeds the range of the reference angle shown by the dotted lines, the position of the bone 301 is corrected to the position at which the angle θ1 is set to the reference angle as illustrated in FIG. 5B. The corrected position is set as a position of the bone 301.

After the bone 301 is fixed at the set position, the position of the bone 302 is calculated according to the external force that the hair 201 including the bone 302 and a portion, which is closer to the tip portion than the bone 302, receives regardless of the presence of the head 202 and body 203. Then, an angle θ2 formed by the bone 302 and the bone 301 is obtained at the calculated position. As illustrated in FIG. 5C, when the calculated angle θ2 exceeds the range of the reference angle shown by the dotted lines, the position of the bone 302 is corrected to the position at which the angle θ2 is set to the reference angle. The corrected position is set as a position of the bone 302.

Similarly, as illustrated in FIG. 5E, the positions of the bones 303 to 305 are sequentially set. Accordingly, the positions of all bones 301 to 305 and 311 to 317 of the player character 200 are set. As illustrated in FIG. 5F, the position of each polygon that forms each portion of the player character 200 is decided using only the positions of the bones 301 to 305 and 311 to 317 as a reference.

The coordinates of the local coordinate system, which show the positions of the bones 301 to 305 and 311 to 317 of the player character 200, are transformed to the coordinates of the world coordinate system. The position of vertexes of the respective polygons, which form the player character 200, can be specified according to the position of the bones 301 to 305 and 311 to 317 in the local coordinate system. The coordinates of the local coordinate system, which show the positions of the vertexes of the respective polygons, are also transformed to the coordinates of the world coordinate system.

When the virtual three-dimensional space is perspective-transformed by the virtual camera, an image of the state that the player character 200 moves on the field in the virtual three-dimensional space is projected on the display screen 122 and recognized by the player. An image, which is projected on a virtual screen from the virtual camera placed in the virtual three-dimensional space, becomes an image that is displayed on the display screen 122. In order to project the image on the virtual screen, a viewing coordinate system is used, and the coordinates of the world coordinate system are transformed to the coordinates of the viewing coordinates.

In order to generate an image projected on the virtual screen by perspective-transformation, there is a need to perform hidden surface removal that removes a surface hidden by another object placed in front. A Z buffer method is used as the hidden surface removal method. After transforming the coordinates of the world coordinate system to the coordinates of the viewing coordinate system, the control section 103 sends the coordinates of each characteristic point to the graphics processor 111 and outputs a drawing instruction thereto. The graphics processor 111 updates the contents of a Z buffer to leave data of a point existing in the front side in connection with each characteristic point based on the drawing instruction. Then, the graphics process 111 develops image data of the relevant characteristic point onto the frame memory 112 every time data of the Z buffer is updated.

The following will explain processing in the three-dimensional video game according to this embodiment. In order to simplify the explanation, it is assumed that an object, which is operated in the virtual three-dimensional space, is only the player character 200. The explanation of processing other than processing particularly relating to the present invention may be omitted. Actually, a vector operation is used to decide the positions of the bones 301 to 303 and 311 to 317 and the polygons. A sine, a cosine, or a tangent are used to determine the angles.

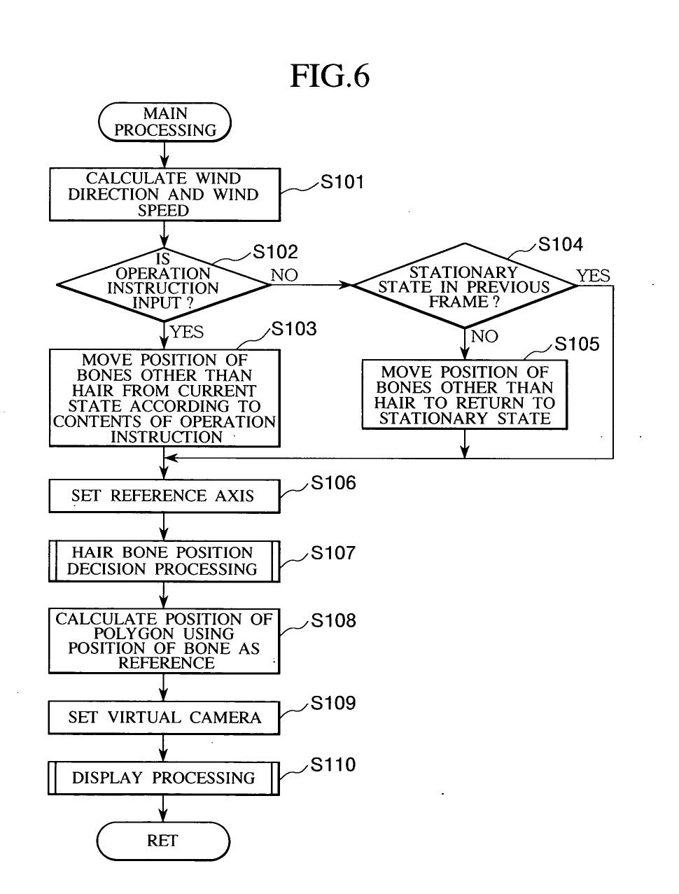

FIG. 6 is a view illustrating a main processing in the three-dimensional video game according to an embodiment. The main processing generates an image corresponding to one frame and is performed by timer interruption every 1/30 second. The main processing may be performed by timer interruption every single field period (1/60 second) or every two frames (1/15 second) depending on the amount of processing.

In the main processing, the control section 103 obtains random numbers for deciding the wind direction and wind speed by executing a random function, and calculates the wind direction and wind speed of the relevant frame according to the obtained random numbers and data of the wind direction and the wind speed relating to several previous frames (step S101). The calculated data of the wind direction and wind speed are stored in a predetermined area of the RAM 105 for the latest several frames.

The control section 103 determines whether an instruction for operating the player character 200 by the player is input from the input section 161 (step S102). When the instruction for operating the player character 200 is not input, the control section 103 determines whether the player character 200 is in a stationary state in the previous frame (step S104). When the player character 200 is in the stationary state, the processing flow proceeds to step S106 directly.

When the instruction for operating the player character 200 is input in step S102, the control section 103 moves the positions (world coordinate system) of the bones 311 to 317 provided to the portions other than the hair 201 of the player character 200 according to the instruction content of the operation from the current state (namely, state according to the bones 311 to 317 decided in the previous frame). In this case, the control section 103 considers the positions of the bones 311 to 317 in the previous several frames in order to express a state that movement of the player character 200 is accelerated (sep S103). Then, the processing flow proceeds to step S106.

When the player character 200 is not in the stationary state in step S104, the control section 103 moves the positions (world coordinate system) of the bones 311 to 317 provided to the portions other than the hair 201 of the player character 200 to return the current state to a predetermined stationary state. In this case, the control section 103 considers the positions of the bones 311 to 317 in the previous several frames to prevent the player from feeling that something is different by stopping the player character 200 rapidly (step S105). Then, the processing flow proceeds to step S106.

In step S106, the control section 103 sets the reference axis 300 for detecting the positions of the bones 301 to 305 provided to the hair 201 based on the positions of the bones 311 and 317 of the player character 200 moved in step S103 or S105 or those that are not moved from the previous frame. When the positions of the bones 311 to 317 provided to the portions other than the hair 201, which is not subjected to the influence of the external force, and the position of the reference axis 300 are detected, the control section 103 performs hair bone position decision processing for detecting the positions of the bones 301 to 305 provided to the hair 201 (step S107).

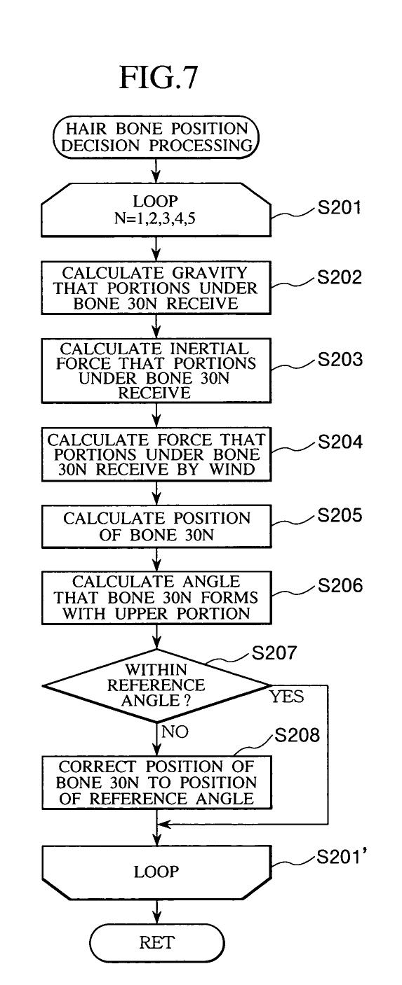

FIG. 7 is a flowchart specifically illustrating the hair bone position decision processing. In order to simplify the explanation, it is assumed that the force applied to the hair 201 of the player character 200 includes only gravity, i.e., the weight of the hair 201, a kinetic inertial force according to the operation of the player character 200 and force received by the wind flowing in the virtual three-dimensional space. In the hair bone position decision processing, the control section 103 performs loop processing by changing a value of N to 1, 2, 3, 4, and 5 sequentially (step S201 to S201′). In the loop, the control section 103 calculates gravity received by portions including bone 30N and thereunder in the hair 201 of the player character 200 (step S202). The control section 103 calculates a kinetic inertial force received by the portions including bone 30N and thereunder in the hair 201 based on the position in the current frame and the position in the previous frame in connection with the bone of the head 202 to which the hair 201 is connected (step S203). The control section 103 calculates force, which the portions including bone 30N and thereunder in the hair 201 receive by the wind flowing in the virtual three-dimensional space, according to the wind speed and wind direction calculated in step S101 (step S204).

The control section 103 calculates a position of the bone 30N (world coordinate system) based on all forces received by the portions including bone 30N and thereunder in the hair 201 calculated in steps S202 to S204. At the time of calculating the position of the bone 30N, reference is made to the position of the bone 30N in the previous frame (step S205). The control section 103 calculates a y component and a z component in a coordinate system where the direction of the reference axis 300 is an x-axial direction in connection with an angle that the bone 30N forms with an upper portion (which is reference axis 300 when the bone 30N is 301 and which is each of bones 301 to 304 when the bone 30N is each of bones 302 to 305) (step S206).

The control section 103 determines whether both y component and z component of the angle that the bone 30N forms with the upper portion are within the range of the reference angle (step S207). When both the y component and z component are within the range of the reference angle and the value of N is not changed up to 5, the control section 103 updates the value of N to perform processing in the loop again. When both the y component and z component are within the range of the reference angle and the value of N is incremented to 5, the processing in this flowchart ends and processing returns to the processing in FIG. 6.

When either the y component or z component of the angle is not within the range of the reference angle, the control section 103 corrects a position (world coordinate system) of the bone 30N to the position where both the y component and z component of the angle that the bone 30N forms with the upper portion are at the reference angle (step S208). When the value of N is not yet incremented to 5, the control section 103 updates the value of N to perform processing in the loop. When the value of N is already incremented to 5, the processing in this flowchart ends and processing returns to the processing in FIG. 6.

Returning to the explanation of FIG. 6, the control section 103 calculates the position (world coordinate system) of the vertex of each polygon that forms each of the portions 201 to 205 of the player character 200 with reference to the positions of the bones 301 to 305 and 311 to 317 of the player character 200 decided in the steps up to S107. More specifically, when the positions of the bones 301 to 305 and 311 to 317 in the local coordinate system are specified, the positions of the vertexes of the respective polygons in the local coordinate system are naturally specified. The control section 103 may transform the coordinates of each polygon in the local coordinate system to the coordinates of the world coordinate system (step S108). There is no need to calculate the positions (world coordinate system) of the vertexes of the polygons that form the portions other than the player character 200 in the virtual three-dimensional space since these positions are fixed.

When the position (world coordinate system) of the vertex of each polygon that forms each of the portions 201 to 205 of the player character 200 is calculated, the control section 103 determines a viewpoint position, a direction of a visual axis, and a magnitude of a visual angle as required, and sets a virtual camera for performing perspective-transformation. The conventional method may be applied to processing for setting the virtual camera (step S109).

When the virtual camera is set and the viewpoint and an optical axis are decided, the control section 103 perspective-transforms the virtual three-dimensional space including the player character onto the virtual screen by the virtual camera, and performs display processing for generating a two-dimensional image to be displayed on the display screen 122 (step S110). When the display processing ends, the control section 103 ends the main processing. The main processing is executed again with start timing of a next frame period.

A brief explanation will be next given of display processing in step S110. Among the coordinates of the vertexes of the respective polygons that form the virtual three-dimensional space including the player character 200, the control section 103 transforms the coordinates of the vertex of each polygon included in a range, which is at least perspective-transformed onto the virtual screen, from the coordinates of the world coordinate system to the coordinates of the viewing coordinate system. The control section 103 sends the coordinates of the vertex of each polygon transformed to the viewing coordinate system to the graphics processor 111, and outputs a drawing command to the graphics processor 111.

In connection with the respective pixels that form the respective surfaces, the graphics processor 111, which has received the drawing command, updates the contents of the Z buffer to leave data of a polygon existing in the front side based on the coordinates of the viewing coordinate system. When the contents of the Z buffer are updated, the graphics processor 111 develops image data of the relevant pixel onto the frame memory 112. The graphics processor 111 also executes processing such as shading, texture mapping and the like to image data to be developed.

The graphics processor 111 reads image data developed onto the frame memory 112, sequentially, and adds a synchronization signal thereto to generate a video signal, and outputs the video signal to the display device 121. The display device 121 displays an image corresponding to the video signal output from the graphics processor 111 on the display screen 122. The image on the display screen 122 is switched every single frame period, so that the player can view the image including the player character 200 moving on the field and the hair 201 moving in response to the external force.

As explained above, in the three-dimensional video game according to this embodiment, the hair 201 of the player character 200 moves under the influence of the external force separately from the operation of the player character 200. However, a magnitude of the received external force differs from portion to portion. Five bones 301 to 305 are provided to the hair 201 and a receiving force for each portion corresponding to each bone is calculated. Accordingly, the amount that the hair 201 is moved in response to the external force for each portion can be reproduced realistically.

If the angle, which each of the bones 301 to 305 forms with the upper portion (reference axis 300 or bones 301 to 304), is calculated based on only the magnitude of the external force received by the hair 201 of the player character 200, any angle may be logically possible. Regarding the angle, which each of the bones 301 to 305 forms with the upper portion, a reference angle at which the hair 201 does not collide with the other portion of the player character is predetermined. The positions of the bones 301 to 305 are corrected to be at the reference angle when the angle that each bone forms with the upper portion is not in the range of the reference angle by the calculation based on the external force. As a result, the positions of the bones 301 to 305 provided to the hair 201 are guaranteed to be located where collisions cannot occur. Thus, such a phenomenon that the polygon forming the hair 201 falls in the other portion of the player character 200 does not occur in the image displayed on the display screen 122.

When the angle, which each of the bones 301 to 305 provided to the hair 201 forms with the upper portion, exceeds the range of the reference angle, the positions of the bones 301 to 305 are corrected to the reference angle. For this reason, when the force applied to the hair 201 is large, it is possible to express movement according to the magnitude of the force as much as possible. Namely, movement of the hair 201 can be expressed to a maximum in the range where no collision occurs, so that a realistic image can be displayed on the display screen 122.

A collision between the hair 201 and the other portion of the player character 200 is determined at the time of drawing the image according to the angle, which each of the bones 301 to 305 forms with the upper portion. This may eliminate the need for using an object for collision detection that is not originally present in the virtual three-dimensional space. The lack of need for the collision detection object can be applied to not only the bone 301 connected to the head 202 but also the bones 302 to 305.

In the video game according to this embodiment, collision detection for preventing collision of the hair 201 is only performed for the bones 301 to 305. If the positions of the bones 301 to 305 provided to the hair 201 are decided, the positions of the vertexes of the respective polygons that form the hair 201 can be decided based on the decided positions of the bones 301 to 305. In other words, since collision detection does not have to be performed for each vertex of the polygon, the amount of calculations required for collision detection can be reduced.

Since the collision detection is not performed for each vertex of the polygon, even if the number of polygons included in the range to be perspective-transformed becomes large, the amount of calculations is not largely increased by the increase in the number of polygons. This makes it possible to reduce the size of each of the polygons that form the object existing in the virtual three-dimensional space including the player character 200. An image, which is perspective-transformed and displayed on the display screen 122, can be a realistic image that expresses a curvature smoothly.

Regarding each of the bones 301 to 305 provided to the hair 201 of the player character 200, such a reference angle at which no collision occurs is predetermined and the position of each of the bones 301 to 305 is decided to be in this range. Accordingly, this makes it possible to appropriately model the operation of an object, such as the hair 201 which is influenced by an external force and which is not a good candidate for calculation based on the inverse kinematics and the inverse dynamics.

In the video game according to this embodiment, since the image is drawn by timer interruption for one frame, drawing processing of the image in the relevant frame must be finished during one frame period. The detection of collisions between the hair 201 and the other portion of the player character is performed only for the positions of the bones 301 to 305, so that a large amount of calculations is not required. Since a large number of polygons can be processed during one frame period, the number of polygons that forms the player character 200 can be relatively increased to reproduce a natural curvature.

The present invention is not limited to the aforementioned embodiment and various modifications and applications may be possible. The following will explain the modification of the above embodiment that is applicable to the present invention.

The aforementioned embodiment explained the case in which the present invention was applied for modeling the operation of the hair 201 of the player character 200. However, the object in which the image is drawn by applying the present invention is not limited to the aforementioned embodiment. If an object is one that is connected to another object and operated independently of the relevant other object at the portion other than the connection point, an image can be drawn for the other kind of object by applying the present invention. Regarding the number of bones included in the object, any number, which is one or more, can be applied. The object may be one that is connected to the other object at two portions. The present invention can be applied to a case in which an operation of a wide object is modeled as well as a narrow object such as hair 201.

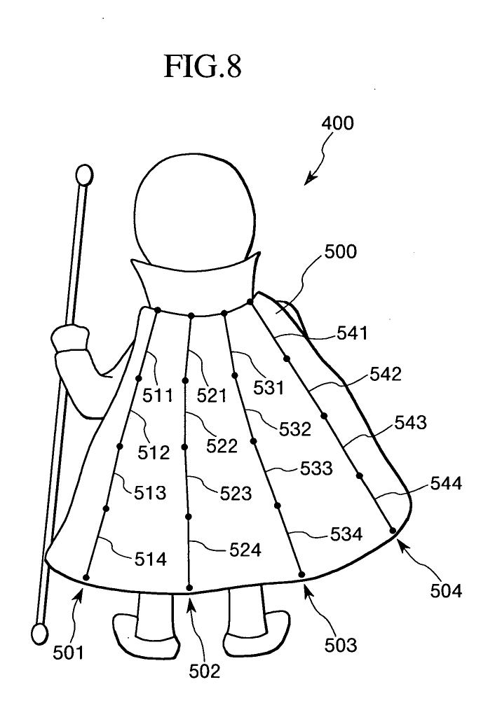

FIG. 8 is a view illustrating a configuration of a character having a wide object. A character 400 wears a cloak as a wide object. Since it is difficult to express an operation of the cloak 500 by only a series of bones, a set of bones 501 including bones 511 to 514, a set of bones 502 including bones 521 to 524, a set of bones 503 including bones 531 to 534, and a set of bones 504 including bones 541 to 544 are provided to the cloak 500. Individual reference axes may be provided to the sets of bones 501 to 504, respectively. A common reference axis may be provided to the sets of bones 501 to 504. The multiple sets of bones 501 to 504 are provided in this way to make it possible to apply the present invention even when an image of the wide object such as the cloak 500 is drawn.

In the aforementioned embodiment, the reference axis 300 was provided separately from the bones 311 to 317 provided to the portions other than the hair 201 of the player character 200. However, the reference axis may be provided to any one of the bones (particularly, the bone 311 of the head 202 to which the hair 201 is connected). In the case where the tip portion of the hair 201 reaches only a certain range of the head 202 without reaching the back of the player character 200, this is especially useful since the reference axis can be easily provided.

In the aforementioned embodiment, the hair 201 of the player character 200 moves under the inertial force of the motion due to the operation of the player character 200, the force of the wind flowing into the virtual three-dimensional space, and the gravity due to its own weight. However, the hair 201 of the player character 200 may move in response to another kind of external force. For example, another character may be operated to hit the player character 200 on the hair 201 with its hand or a weapon. In this case, determination of the collision with another character's hand or weapon can be executed with reference to the positions of the bones 301 to 305.

The aforementioned embodiment explained the case in which the present invention was applied at the time of drawing the image of the object, which was passively operated by the external force. However, the present invention can be applied at the time of drawing the image of the object, which operates actively as in the character's hand and leg, and the image of the object, which operates actively and is operated passively by the external force.

The aforementioned embodiment was explained on the assumption that the bones included in the object (specifically, the hair 201) were linearly shaped. However, curved bones may be included in the object. An angle, which each bone forms with the reference axis or the upper portion, can be calculated by a tangent at both ends of each bone.

In the aforementioned embodiment, it was determined whether the hair 201 collided with the other portion of the player character 200 depending on whether the angle, which each of the bones 301 to 305 provided to the hair 201 formed with the upper portion, was in the range of the reference angle. The collision detection can be actually performed for only the bones 301 to 305 provided to the hair 201 using the object for collision detection. After the positions of the bones 301 to 305 are given by the detection result using the object for collision detection, the position of each polygon may be decided according to only the positions of the given bones 301 to 305.

In the aforementioned embodiment, the video game apparatus 100, which was a special-purpose machine, was used as a platform. In contrast to this, any apparatus such as a general-purpose computer may be used if the apparatus includes the same structural components as those of the video game main body 101 and a function of drawing a three-dimensional image. Moreover, a portable video game apparatus, which contains the display device 121 and the sound output device 125 in the same cabinet as that of the video game main body 101, may also be used.

A semiconductor memory card may be used as the storage medium 131 in place of a DVD-ROM or CD-ROM. In the video game apparatus main body 101 or the portable game apparatus, a card slot for inserting the memory card may be formed in place of the DVD/CD-ROM drive 113. In the case of the general-purpose personal computer, the program and data relating to the present invention may be prestored to the HDD 107 instead of being stored to the storage medium 131. Regarding the storage medium for storing the program and data relating to the present invention, any storage medium may be used according to the physical form of hardware and the distribution thereof.

The program for executing the video game of the present invention may be stored on a fixed disc apparatus provided in a Web server apparatus existing on the network 151. The Web server apparatus may convert the program and data stored in the fixed disc apparatus to a signal and superimpose the signal on a carrier wave, and distribute it to the video game main body 101 via the network 151 and the communications medium 141. The program, which the communications interface 115 received from the Web server apparatus, can be stored in the HDD 107 and loaded to the RAM 105 at an executing time.

The aforementioned embodiment explained the case in which the present invention was applied to draw the image of the game in the three-dimensional video game. However, the present invention can be used in the field of the computer graphics processing for drawing a three-dimensional image without being limited to the three-dimensional video game. The present invention is particularly suitable for use in the field of the three-dimensional computer graphics processing for drawing a three-dimensional image with real time. Even if this is not one that is applied as a part of the program of the video game, a program including a function of drawing the three-dimensional image as mentioned above can be distributed by the same method as that of the program for the video game.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in its aspects. Although the invention has been described with reference to particular means, materials and embodiments, the invention is not intended to be limited to the particulars disclosed; rather, the invention extends to all functionally equivalent structures, methods, and uses such as are within the scope of the appended claims.

1. A three-dimensional image drawing apparatus that draws a three-dimensional image by modeling a first object and a second object that is connected to the first object and includes a bone defining display positions of multiple polygons, the second object moving independently of the first object at a portion other than a connection point, comprising:
a bone position calculator that calculates a position of the bone included in the second object when the second object moves according to a predetermined operation condition;
a collision detector that determines whether the second object is where no collision with the first object occurs, based upon the calculated position of the bone;
a bone position corrector that recalculates the position of the bone included in the second object to be where no collision with the first object occurs when the second object is determined as being where collision with the first object occurs;
a polygon position calculator that calculates a position of each of the multiple polygons that form the second object with reference to the position of the bone, which is located where no collision with the first object occurs; and
an image rendering system that draws the multiple polygons that form the second object according to the position calculated by the polygon position calculator to create an image where the first and second objects are modeled.

2. The three-dimensional image drawing apparatus according to claim 1, wherein the collision detector includes an angle determiner that determines whether an angle, which the bone included in the second object forms with a reference axis set with reference to the first object, is within a range of a reference angle predetermined as an angle at which no collision with the first object occurs, and determines that the second object is where no collision with the first object occurs when the angle determiner determines that the angle is within the range of the reference angle.

3. The three-dimensional image drawing apparatus according to claim 2, wherein the bone position corrector recalculates the position of the bone included in the second object to be where the angle matches the reference angle when the angle determiner determines the angle is not within the range of the reference angle.

4. The three-dimensional image drawing apparatus according to claim 2, wherein the second object is passively moved in response to a force applied externally.

5. The three-dimensional image drawing apparatus according to claim 1,
wherein the second object includes a first bone that is directly connected to the first object and a second bone that is directly connected to the first bone and is not directly connected to the first object; and
wherein the collision detector includes:
a first angle determiner that determines whether an angle, which the first bone forms with a reference axis, is within a range of a first reference angle;
a second angle determiner that determines whether an angle, which the second bone forms with the first bone, is within a range of a second reference angle; and
determines that the second object is where no collision with the first object occurs when the first and second angle determiners determine that the angles are within the first and second angle ranges, respectively.

6. The three-dimensional image drawing apparatus according to claim 5, wherein the bone position calculator includes:
a first bone position calculator that calculates a position of the first bone according to a force applied to portions corresponding to the first bone and the second bone of the second object; and
a second bone position calculator that calculates a position of the second bone according to a force applied to a portion corresponding to the second bone of the second object.

7. The three-dimensional image drawing apparatus according to claim 6, wherein the second bone position calculator calculates the position of the second bone with reference to the recalculated position of the first bone when the position of the first bone is recalculated.

8. The three-dimensional image drawing apparatus according to claim 1,
wherein the first object comprises multiple polygons and includes at least one bone that defines the display positions of the multiple polygons;
wherein the apparatus further comprises:
a first object bone position calculator that calculates a position of the at least one bone included in the first object when the first object moves according to a predetermined operation condition different from the operation condition of the second object; and
a reference axis setting section that sets a reference axis according to the position of the at least one bone included in the first object; and
wherein the collision detector determines whether the second object is where no collision with the first object occurs according to a positional relationship between the bones included in the second object and the reference axis.

9. The three-dimensional image drawing apparatus according to claim 8, further comprising:
a first object polygon position calculator that calculates positions of the multiple polygons that form the first object with reference to the position of the at least one bone included in the first object,
wherein the image rendering system further draws the multiple polygons that form the first object according to the position calculated by the first object polygon position calculator to create an image where the first and second objects are modeled.

10. The three-dimensional image drawing apparatus according to claim 1, further comprising:
an interruption generator that generates an interruption for each frame period or each field period,
wherein the bone position calculator, the collision detector, the bone position corrector, the polygon position calculator, and the image rendering system start in response to the generated interruption.

11. A three-dimensional image drawing apparatus that draws a three-dimensional image by modeling a first object, comprising multiple polygons, and a second object, comprising multiple polygons and includes a bone defining display positions of the multiple polygons, the second object moving independently of the first object at a portion other than a connection point, the three-dimensional image drawing apparatus having a program memory that stores a program and a processor that executes the program, wherein the program causes the processor to execute:
calculating the position of the bone included in the second object when the second object moves according to a predetermined operation condition;
determining whether the second object is where no collision with the first object occurs, based upon the calculated position of the bone;
recalculating the position of the bone included in the second object to be where no collision with the first object occurs when the second object is determined as being where collision with the first object occurs;
calculating the position of each of the multiple polygons that form the second object with reference to the position of the bone, which is located where no collision with the first object occurs; and
drawing the multiple polygons that form the second object according to the calculated position to create an image where the first and second objects are modeled.

12. The three-dimensional image drawing apparatus according to claim 11,
wherein the first object further comprises at least one bone that defines the display positions of the multiple polygons; and
wherein the program causes the processor to execute:
calculating the position of the at least one bone included in the first object when the first object is moved according to a predetermined operation condition different from the operation condition of the second object;
calculating positions of the multiple polygons that form the first object with reference to the position of the at least one bone included in the first object; and
drawing the multiple polygons according to the calculated positions of the multiple polygons that form the first and second objects to create an image where the first and second objects are modeled.

13. The three-dimensional image drawing apparatus according to claim 12,
wherein the processor includes a first processor and a second processor that executes the program in accordance with a command from the first processor;
wherein the program causes the first processor to execute:
calculating the position of the bone included in the second object;
determining whether the position of the bone included in the second object is where no collision with the first object occurs;
recalculating the position of the bone included in the second object to be where no collision with the first object occurs when the second object is determined as being where collision with the first object occurs;
calculating the positions of the multiple polygons that form the second object;
calculating the position of the at least one bone included in the first object;
calculating the positions of the multiple polygons that form the first object; and
outputting a drawing command with the positions of the multiple polygons that form the first and second objects to the second processor as a step of drawing the image where the first and second objects are modeled;
wherein the program causes the second processor to execute creating the multiple polygons according to the position output from the first processor based on the drawing command from the first processor to create an image where the first and second objects are modeled.

14. A method for drawing a three-dimensional image by modeling a first object and a second object that is connected to the first object and includes a bone defining display positions of multiple polygons, the second object moving independently of the first object at a portion other than a connection point, comprising:
calculating a position of the bone included in the second object when the second object moves according to a predetermined operation condition;
determining whether the second object is where no collision with the first object occurs, based upon the calculated position of the bone;
recalculating the position of the bone included in the second object to be where no collision with the first object occurs when the second object is determined as being where collision with the first object occurs;
calculating a position of each of the multiple polygons that form the second object with reference to the position of the bone, which is located where no collision with the first object occurs; and
drawing the multiple polygons that form the second object according to the calculated position to create an image where the first and second objects are modeled.

15. A computer-readable storage medium storing a program for drawing a three-dimensional image by modeling a first object and a second object that is connected to the first object and includes a bone defining display positions of multiple polygons, the second object moving independently of the first object at a portion other than a connection point, the program causing a computer apparatus to execute:
calculating a position of a bone included in the second object when the second object moves according to a predetermined operation condition;
determining whether the second object is where no collision with the first object occurs, based upon the calculated position of the bone;
recalculating the position of the bone included in the second object to be where no collision with the first object occurs when the second object is determined as being where collision with the first object occurs;
calculating a position of each of the multiple polygons that form the second object with reference to the position of the bone, which is located where no collision with the first object occurs; and
drawing the multiple polygons that form the second object according to the calculated position to create an image where the first and second objects are modeled.

16. A carrier wave superimposed on a data signal of a program for drawing a three-dimensional image by modeling a first object and a second object that is connected to the first object and includes a bone defining display positions of multiple polygons, the second object moving independently of the first object at a portion other than a connection point, wherein the program causes a computer apparatus to execute:
calculating a position of a bone included in the moving second object when the second object moves according to a predetermined operation condition;
determining whether the second object is where no collision with the first object occurs, based upon the calculated position of the bone;
recalculating the position of the bone included in the second object to be where no collision with the first object occurs when the second object is determined as being where collision with the first object occurs;
calculating a position of each of the multiple polygons that form the second object with reference to the position of the bone, which is located where no collision with the first object occurs; and
drawing the multiple polygons that form the second object according to the calculated position to create an image where the first and second objects are modeled.
